(19) United States
(12) Patent Application Publication
(10) Pub. No.: US 2021/0151175 A1
(43) Pub. Date: May 20, 2021

(54) SYSTEMS AND METHODS INDICATING PENDING INFORMATION FOR PATIENTS

(71) Applicant: GE Precision Healthcare LLC, Wauwatosa, WI (US)

(72) Inventor: Isani Mishra, Bengaluru (IN)

(73) Assignee: GE Precision Healthcare LLC, Wauwatosa, WI (US)

(21) Appl. No.: 16/688,526

(22) Filed: Nov. 19, 2019

(51) Int. Cl. G16H 40/63; G16H 10/60; G16H 10/40

(52) U.S. Cl. CPC G16H 40/63; G06F 3/04842; G16H 10/40; G16H 10/60

(57) ABSTRACT

Systems and methods for providing information to a caregiver, including displaying with an interface real-time data for a patient receiving care from the caregiver, and providing access to a results database configured to store results data for the patient. The method further includes determining whether a test for the patient is pending, where upon completion of the test a test result from the test is stored among the results data in the results database. The method further includes displaying, when the test is determined to be pending, a visual indicator on the interface in addition to the real-time data, where the visual indicator provides the information to the caregiver that the test is pending and that the test result corresponding thereto will be accessible via the results database only after the test is completed.

### FIELD

The present disclosure generally relates to systems and methods for indicating pending information for patients, and more particularly to systems and methods for indicating pending information for patients by including a visual indicator thereof.

### BACKGROUND

Clinical monitoring and decision dashboards are known in the art for monitoring the status of one or more patients, such as within an Intensive Care Unit (ICU), for example. These dashboards also provide for remote monitoring of one or more patients by a caregiver not on the premises. Information generally provided on these dashboard includes identifying information for the patient, such as name, date of birth, and patient identification number, various diagnosis and gradings, such as a likelihood of developing sepsis, and/or real-time or historic snapshots of various health metrics such as heartrate, blood pressure, ECG data, temperature and/or the like. One such exemplary dashboard is the GE Decisio platform.

### SUMMARY

This Summary is provided to introduce a selection of concepts that are further described below in the Detailed Description. This Summary is not intended to identify key or essential features of the potentially claimed subject matter, nor is it intended to be used as an aid in limiting the scope of the potentially claimed subject matter.

One embodiment of the present disclosure generally relates to a method for providing information to a caregiver. The method includes displaying with an interface real-time data for a patient receiving care from the caregiver, and providing access to a results database configured to store results data for the patient. The method further includes determining whether a test for the patient is pending, where upon completion of the test a test result from the test is stored among the results data in the results database. The method further includes displaying, when the test is determined to be pending, a visual indicator on the interface in addition to the real-time data, where the visual indicator provides the information to the caregiver that the test is pending and that the test result corresponding thereto will be accessible via the results database only after the test is completed.

Another embodiment generally relates to a method for providing information to a caregiver. The method includes displaying with an interface real-time data for a patient receiving care from the caregiver, and providing access to a results database configured to store results data for the patient. The method further includes determining a quantity of tests and types of individual tests within the tests pending for the patient, where upon completion of each of the individual tests a test result corresponding thereto is stored among the results data in the results database. The method further includes displaying, when the quantity of tests pending is determined to be at least one, a visual indicator on the interface in addition to the real-time data, where the visual indicator includes the quantity of the tests and types of each of the individual tests therein determined to be pending, and where the visual indicator provides the information to the caregiver that the test results for each of the individual tests will be accessible via the results database only as each of the individual tests is completed. The method further includes displaying the types of each of the individual tests are displayed only when the caregiver at least one of hovers over and clicks on the visual indicator with a selection device.

Another embodiment generally relates to a method for providing information to a caregiver. The method includes displaying with an interface real-time data for a patient receiving care from the caregiver, where the interface is configured to concurrently display additional data corresponding to other patients receiving care from the caregiver. The method further includes providing access to a results database configured to store results data for the patient and determining a quantity of tests and types of individual tests within the tests pending for the patient, where upon completion of each of the individual tests a test result corresponding thereto is stored among the results data in the results database. The method further includes determining an expected completion time for each of the individual tests determined to be pending, where the excepted completion time is when the test result is expected to be stored among the results data in the results database. The method further includes displaying, when the quantity of tests pending is determined to be at least one, a visual indicator on the interface in addition to the real-time data, where the visual indicator includes the quantity of the tests and types of each of the individual tests therein determined to be pending, where the visual indicator provides the information to the caregiver that the test results for each of the individual tests will be accessible via the results database only as each of the individual tests is completed. The method further includes displaying the types of each of the individual tests are displayed only when the caregiver at least one of hovers over and clicks on the visual indicator with a selection device, and displaying the expected completion time for each of the individual tests determined to be pending only when the caregiver with a selection device at least one of: hovers over the visual indicator, clicks on the visual indicator, and clicks on the type corresponding to at least one of the individual tests being displayed on the interface.

Various other features, objects and advantages of the disclosure will be made apparent from the following description taken together with the drawings.

### BRIEF DESCRIPTION OF THE DRAWINGS

The present disclosure is described with reference to the following Figures.

FIG. 1A is an exemplary interface incorporating the systems and methods for providing information to the caregiver according to the present disclosure;

FIGS. 1B-1D depict close ups of the interface shown in FIG. 1a, whereby FIG. 1d depicts a first tier display of the presently disclosed visual indicator;

FIG. 2 depicts an interface similar to that shown in FIG. 1d now displaying a second tier display as the visual indicator;

FIG. 3 is depicts an interface similar to that shown in FIG. 1d now displaying a third tier display as the visual indicator;

FIG. 4 depicts and exemplary process flow for method of providing information to the caregiver according to the present disclosure;

FIGS. 5A-5C depict alternate embodiments for visual indicators such as may be incorporated within the second tier display shown in FIG. 2 and FIG. 6 is a schematic view of an exemplary control system for providing information to the caregivers according to the present disclosure.

### DETAILED DISCLOSURE

A typical patient dashboard concurrently displays a wide variety of information for both a single patient and multiple patients such that a caregiver can simultaneously manage multiple caseloads, for example within an Intensive Care Unit (ICU). It is through this dashboard that caregivers may review and integrate patient data from multiple sources to determine an appropriate course of treatment. For clarity, the data sources are generally referred to herein as originating in the Electronic Medical Records (EMR) system. However, other sources of data may also be integrated according to the present disclosure, including data stored within a Lab Information System (LIS), a Picture Archiving and Communication System (PACS), a Clinical Information System (CIS), and/or another databases and workflow systems.

The inventor has identified problems with patient dashboards presently known in the art. For example, the information provided on a current dashboard and through the various data sources (e.g., an EMR system) is only visible to the caregiver after a test or other inquiry is finalized and the corresponding results are published and saved in the record. In other words, systems presently known in the art do not provide a means for communicating to the caregiver that a work is in progress and that new corresponding information will then become available. This work may include a test that has been ordered, is currently being conducted, or is awaiting finally analysis by a specialist, for example. These tests or other inquiries are also referred to herein as activities. This lack of information is particularly exacerbated in the context of the caregiver that is a remote consultant, specialist, or tele-ICU specialist, whereby no personal interactions occur between the remote caregiver and the patient or other clinicians. In this case, the remote caregiver can only refer to the dashboard and results published in the various data sources when making clinical decisions.

Since caregivers are not currently informed about any investigations that are in progress, including tests or other procedures ordered by other physicians or specialists, this insight cannot be incorporated within clinical decisions and treatment management for the patient. If instead the caregiver had an indication that new data is pending, that caregiver could decide to wait until the latest information becomes available before finalizing a course of action, providing for a more informed decision for the patient. Similarly, the present lack of awareness of pending activities leads to duplication in ordering investigations or reinstating investigation orders to floor clinicians who may have already executed an initial process lab sampling, for example. Providing the caregiver with an indication of pending activities therefore cuts down on redundant or wasted workflow between the specialist and the floor clinician, saving time and medical resources.

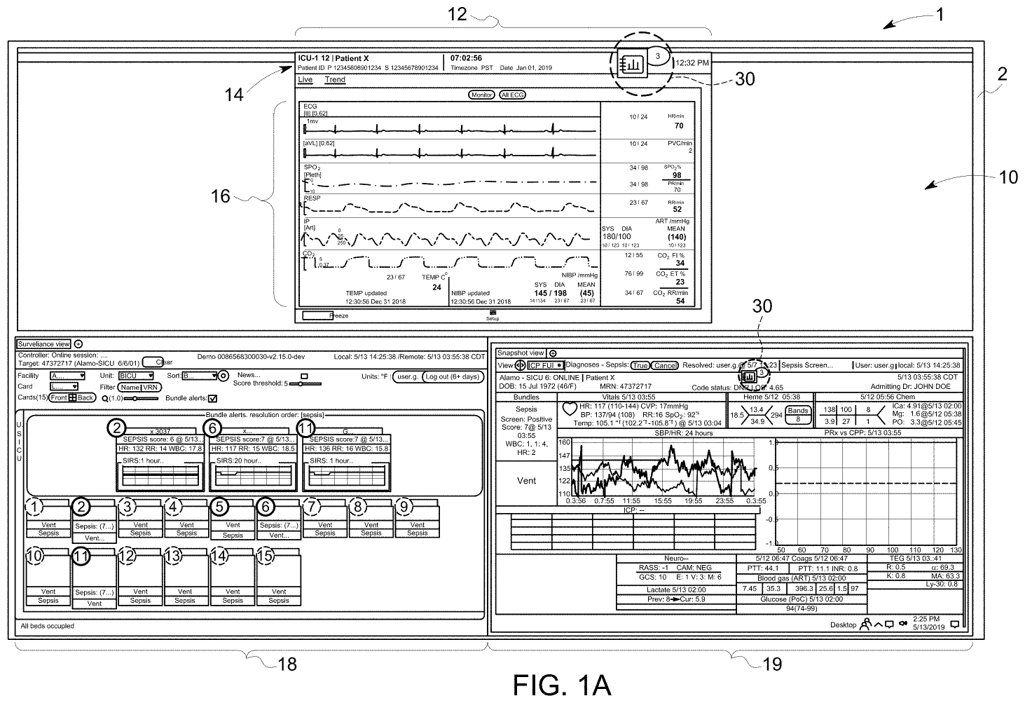

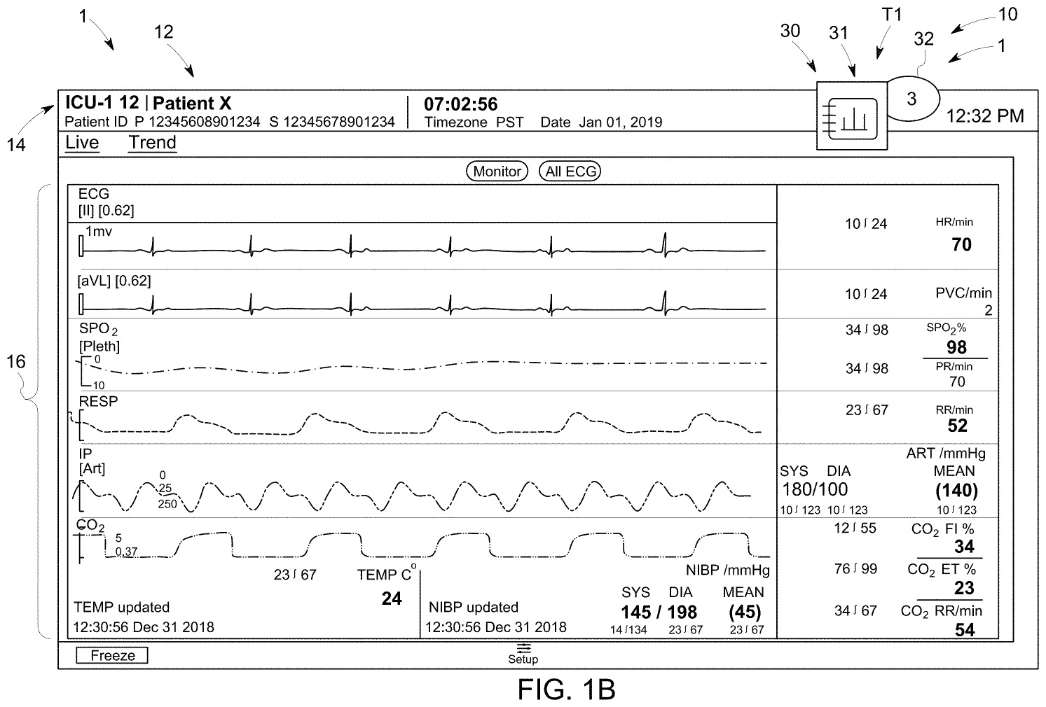

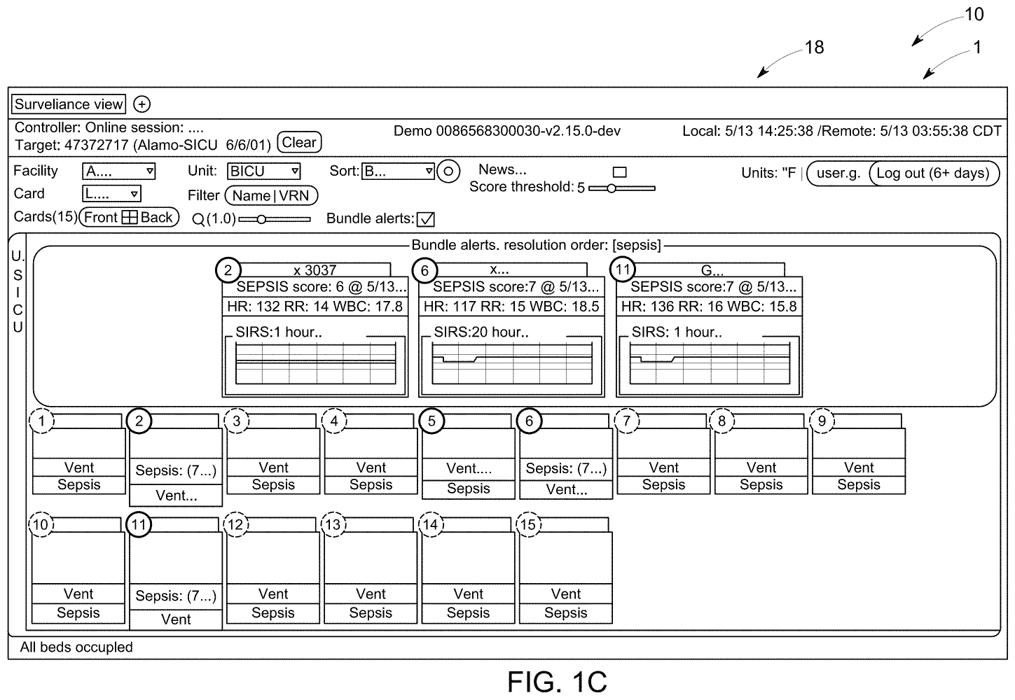

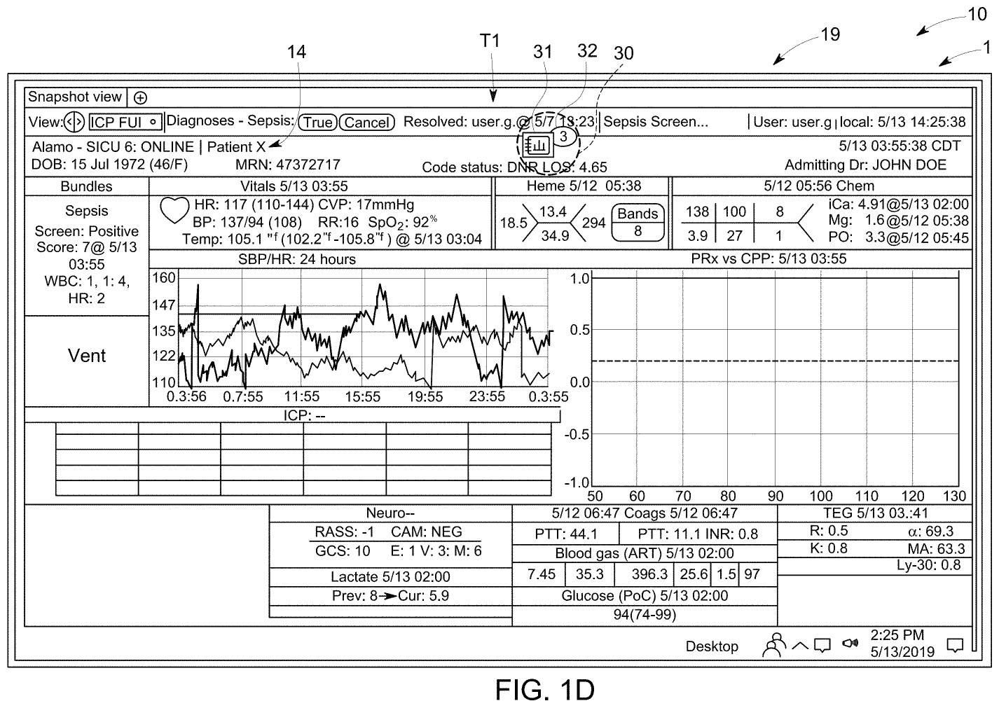

FIG. 1A depicts an exemplary system 1 for providing additional information to a caregiver according to the present disclosure. The system 1 includes a display device 2, such as a computer monitor, that displays an interface 10 thereon. In the example shown, the interface 10 includes a single patient display region 12 as well as a multiple patient display region 18 and a snapshot display region 19. The snapshot display region 19 in the present example shows data corresponding to the same patient as that of the single patient display region 12, but provides historical data and analysis summaries, whereas the single patient display region 12 in the present example shows real-time data 16, such as ECG, SPO₂, CO₂, heartrate, and other philological data. However, the particular types of data displayed may vary, as well as how the data is divided into one or more regions. In the example shown, additional information is also provided in the single patients display region 12, such as patient information 14. As will be discussed below, a visual indicator 30 as provided by the present disclosure is also shown in both the single patient display region 12 and the snapshot display region 19 corresponding to that same patient.

FIGS. 1B-1D depict close up view of the exemplary interface 10 shown in FIG. 1A, and particularly the single patient display region 12, multiple patient display region 18 and snapshot display region 19, respectively. FIGS. 1B and 1D depict a visual indicator 30 according to the present disclosure, which provides an indication to the caregiver that one or more activities are in process, but do not yet have published results available for review. In the examples of FIGS. 1B and 1D, the visual indicator 30 is shown as a first tier display T1, which includes an icon 31 that is displayed when activities are pending. In certain embodiments, no visual indicator 30 is shown when there are presently no pending activities. In the example shown, the visual indicator 30 further includes a quantity 32, which shows the number of tests or other procedures that are pending, which in the present example is three. It will be recognized that as tests are finished and the results become published, this quantity 32 is updated, until the visual indicator 30 fully disappears when the quantity 32 reaches zero.

In addition to indicating whether any activities are pending, and the quantity 32 thereof, the presently disclosed systems and methods allow the caregiver to determine the specific nature and timing of such activities. This additional information is available as a summary view, and as a detailed view, which is discussed further below. In certain embodiments, the system 1 is configured to provide the summary view (as a second tier display T2) when the caregiver hovers over the first tier display T1 of the visual indicator 30, and the detailed view (as a third tier display T3) when the caregiver selects (e.g., clicks on) the first tier display T1 or the second tier display T2.

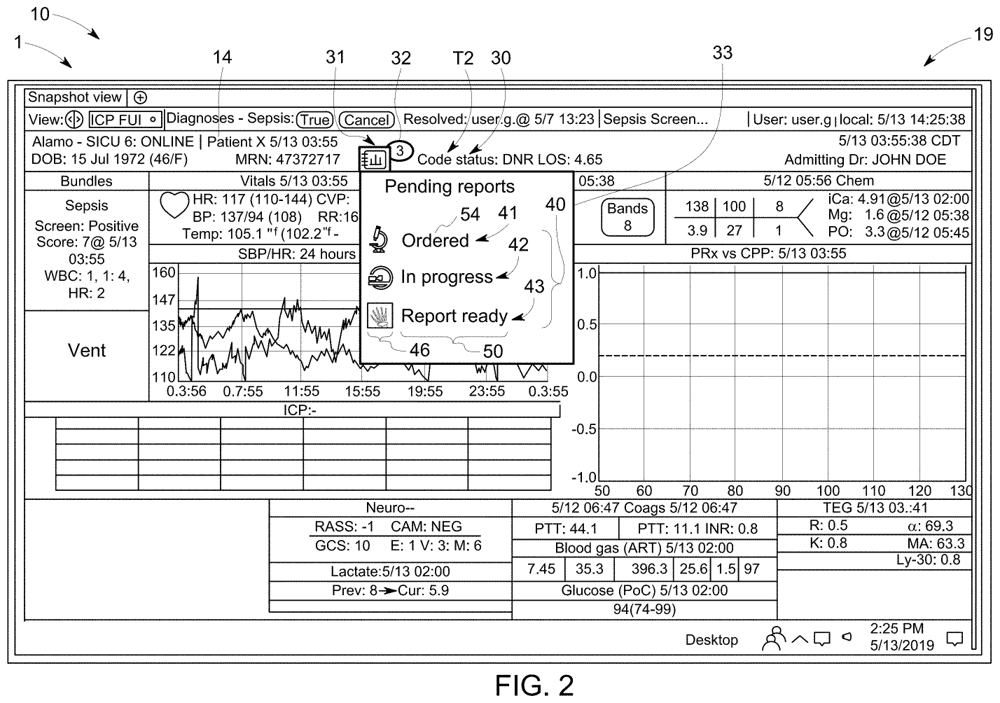

FIG. 2 depicts the snapshot display region 19 from FIG. 1D, but now with the visual indicator 30 shown as a second tier display T2. As shown, the second tier display T2 includes the icon 31 from the first tier display T1, but adds to it a summary data box 33. In the example shown, the summary data box 33 shows which types 40 of tests or other investigations are presently pending, which in the present case are a first type 41, a second type 42, and a third type 43. Other numbers of types 40 may also be displayed at once, and the classification and labeling thereof may vary according to the particular context for using the system 1. Each of the types 40 may also be listed in written form and/or displayed as a symbol 46 corresponding thereto.

As shown in the example of FIG. 2, the first type 41 of the types 40 of activities indicated as pending corresponds to laboratory tests, the second type 42 corresponds to volumetric medical imaging (such as a CT scan), and the third type 43 corresponds to x-ray imaging. As also shown in summary data box 33, each of the types 40 is provided with a corresponding status indication 50, such as text 54, describing the status of that particular type 40 of pending activity. The present example shows that orders have been placed for the first type 41, a second type 42 is in progress, and reports for the third type 43 are ready, but have not yet been published for viewing (e.g. through the EMR after being read by a specialist. It should be recognized that multiple entries may be shown for a same type 40 of activity, including the status indications 50 corresponding thereto.

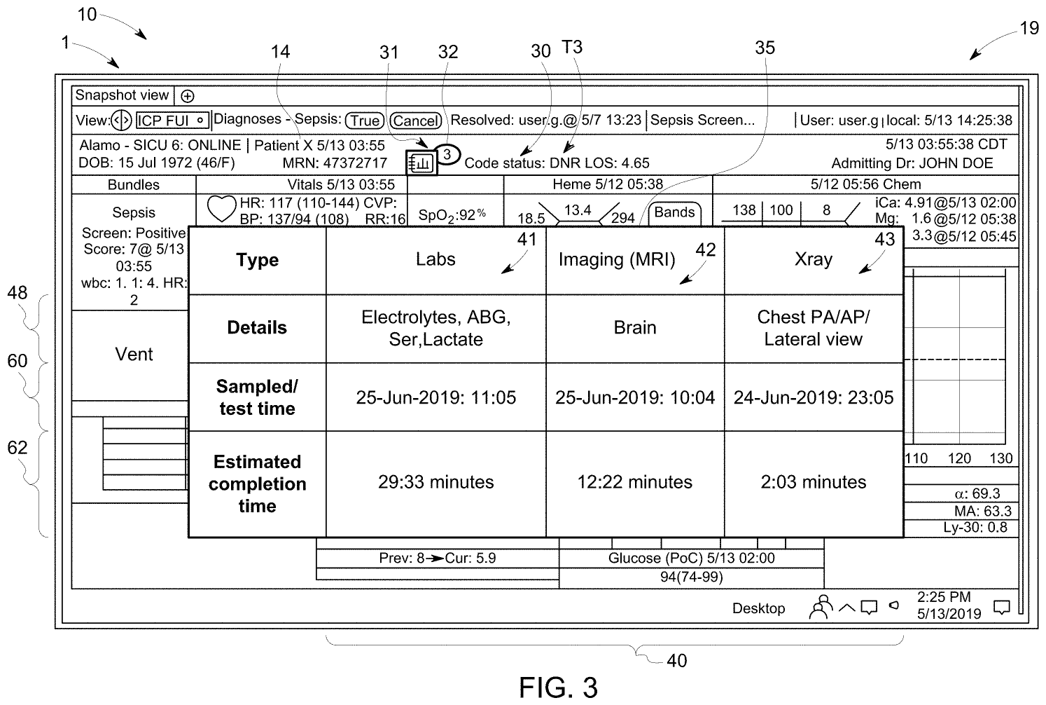

FIG. 3 shows a third tier display T3 for the visual indicator 30, which for example is accessed by clicking on the icon 31 when the visual indicator 30 is displayed as a first tier display T1, or clicking the icon 31 or summary data box 33 when the visual indicator 30 is shown as a second tier display T2. In the third tier display T3, a detailed data box 35 is provided in place of the summary data box 33 of the second tier display T2. In the present example, the icon 31 associated with the first tier display T1 remains displayed. The detailed data box 35 provides additional details 48 for what activities are in progress, as well as start time and date information 60 for starting the activity (e.g., a sample time for a blood test). The detailed data box 35 also provides an estimated completion time 62 for each activity.

In the example shown, each of the columns responds to one of the types 40 for tests that are presently pending, shown here with Labs as the first type 41, Imaging (MRI) as the second type 42, and X-ray imagining as the third type 43. The rows within the detailed data box 35 then corresponds to the additional details 48, start time and date information 60, and estimated completion time 62 corresponding to each of the types 40 of activities. For example, the additional details 48 corresponding to the first type 41 of testing (labs) reveals that the particular testing in process is investigating the patient's electrolytes, ABG, Ser. (serology), and lactate levels, and that the corresponding start time and date information 60 was 11:05 am on Jun. 25, 2019, with an expected completion time in 29 minutes and 33 seconds.

As will be discussed further below, the estimated completion time 62 may be based on standard times data 94 contained within a results database 90 (which may be stored within an EMR database) corresponding to each possible activity. This expected completion time may be periodically updated based on progress made in the corresponding work flow for that activity, which may be restored as results data 92 within the results database 90. The estimated completion time 62 may also be updated based on actual times for other tests that day (which may also be stored in the results database 90), the patient's position in a queue, or other sources for improving the accuracy of predicting the estimated completion time 62.

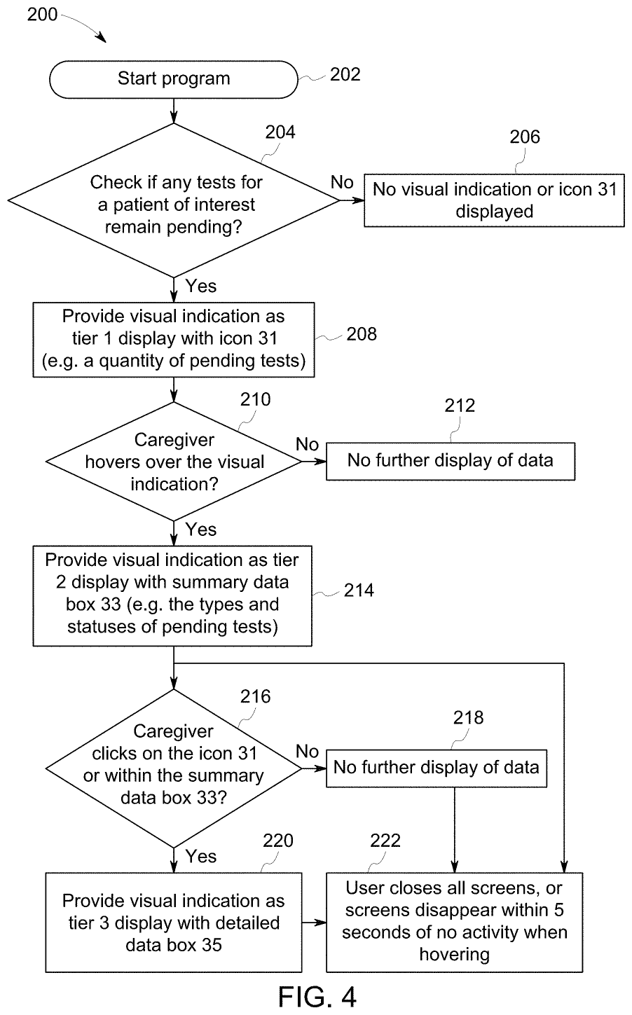

FIG. 4 depicts an exemplary process flow for providing information to the caregiver according to the present disclosure. The method 200 begins by starting the program in step 202, which as previously described may be similar to a clinical dashboard presently known in the art. The system 1 (through a control system 100, discussed below) then determines in step 204 whether any activities for a particular patient of interest remain pending, such as by engaging with a results database 90. If there are no pending activities identified in step 204, no visual indication 30 is displayed on the interface 10. If alternatively it is determined in step 204 that at least one activity is pending, a visual indication 30 is provided in step 208 (such as a first tier display T1) with an icon 31 on the interface 10.

In certain embodiments, the visual indicator 30 further includes a quantity 32 of the number of activities that are pending, as discussed above. In step 210, it is determined whether a caregiver has hovered over the visual indication 30 displayed on the interface 10. If the caregiver has not hovered over the icon 31, no further display of data is provided and the interface 10 continues to display the first tier display T1 of the visual indicator 30. If alternatively the caregiver is determined to be hovering over the visual indication 30 in step 210, step 214 provides the visual indication 30 as a second tier display T2 that includes the summary data box 33 discussed above. This may include the particular types 40 and status indications 50 for the tests determined to be pending, as described above with respects to FIG. 2, for example.

In the embodiment shown, it is next determined in step 216 whether the caregiver not only hovers over the icon 31, but clicks on either the icon 31 or the summary data box 33 being displayed. If the caregiver is not determined to have clicked on the icon 31 or summary data box 33 in step 216, no further display of data is made on the interface 10, and in certain embodiments all screens (e.g., the summary data box 33) will disappear within 5 seconds if no additional activity or movement is detected. Alternatively, if it is determined in step 216 that the caregiver has clicked on the icon 31 or the summary data box 33, step 220 provides for the visual indication 30 to be shown as a third tier display T3, displaying the detailed data box 35 discussed above. From there, the user may manually close he screens, or the screens may time out after some duration of inactivity is detected (optional step 222).

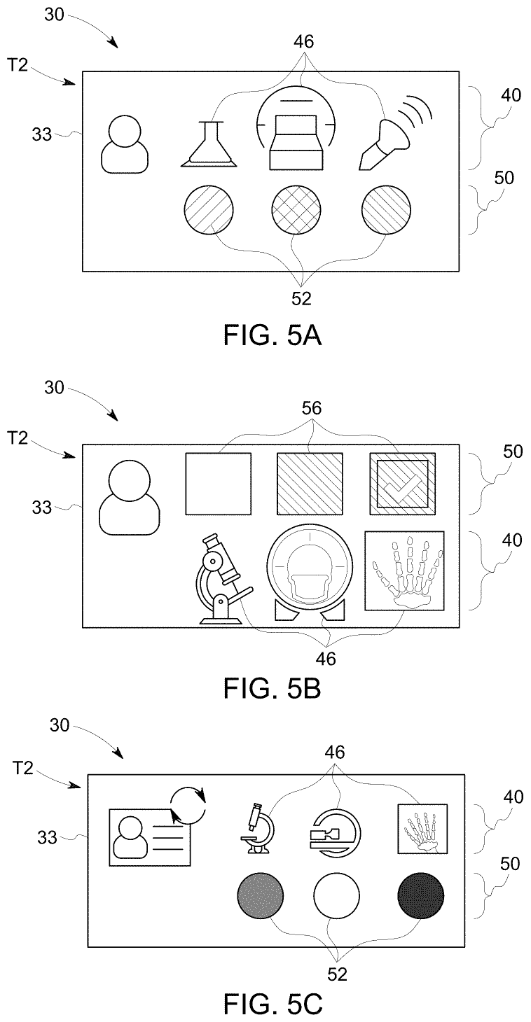

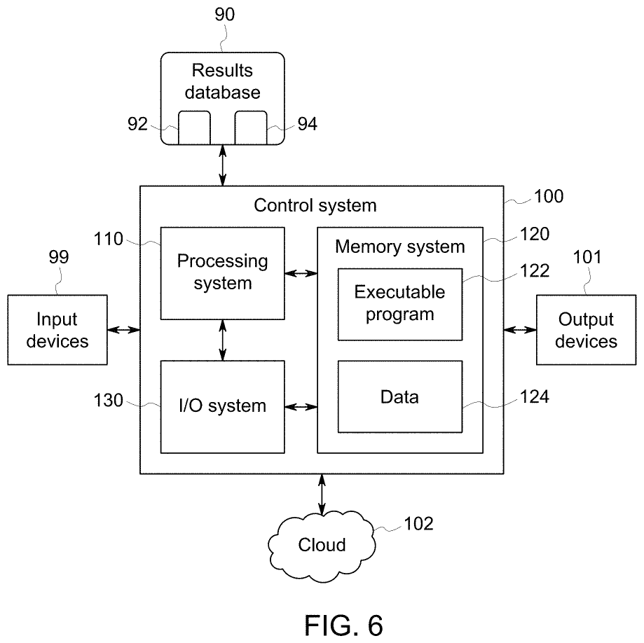

FIGS. 5A-5C depict alternative embodiments of symbols 46 for the types 40 of activities that are determined to be pending, as well as color indicators 52 and symbols 56 corresponding to status indicators 50 for these activities. In the examples shown, each of the symbols 46 and status indicators 50 are shown within a summary data box 33 corresponding to a second tier display as discussed above. In FIG. 5A, the symbols 46 correspond to lab results, imaging (such as MRI), and ultra sound, respectively from left to right after a patient image. The corresponding color indicators 52, which are shown here in hashed lines to represent different colors such as green, yellow, and red, indicate the status of each type 40. In certain examples, this status is based in part of the expected completion time 62 corresponding to that type 40. FIGS. 5B and 5C depict further alternate symbols 46 for the same or alternate types 40 of tests, as well as different symbols 56 and color indicators 52 for showing the corresponding statuses has status indicators 50. In the embodiment of FIG. 5B, the symbols 56 are boxes shown to be open when a test is ordered but has not yet been started, shown with hashed lines when a test is in process, and shown with a check mark when a test has been completed but has not yet been published to the EMR for review (e.g., if the test is awaiting review by a specialist). Once results are published, the type 40 would no longer be displayed in the summary data box 33. Likewise, in the example of 5C, the color indicators 52 corresponding to the types 40 are not different colors, but different greyscale shades depending on the status for the particular type 40 of activity pending.

FIG. 6 depicts an exemplary control system 100 for providing the systems and methods described herein. As shown, the control system 100 is provided with various input devices 99, such as a mouse for navigating on the interface 10. Likewise, the control system 100 includes output devices 101, such as the display device 2 for displaying the interface 10. A results database 90, which corresponds to both an input device 99 and an output device 101, is also provided in communication with the control system 100. In the example shown the result database 90 includes results data 92, which provides the information provided in the summary data box 33 associated with the second tier data T2, as well as the detailed data box 35 associated with the third tier display T3 of the visual indicator 30. This results data 92 may ultimately also contain the completed and published results from tests that are readable by the caregiver, which before completion are foreshadowed to the caregiver via the visual indicator 30 as previously described. The results database 90 may also include standard times data 94 as discussed above, which may include standard expectations of completion times based on the type 40 of test, the schedule of the lab, imaging site, and/or reviewing specialist, or other factors impacting the accuracy of expected completion time 62.

Additional information detailing an exemplary control system 100 such as shown in FIG. 6 is provided herein. It should be recognized that certain aspects of the present disclosure are described or depicted as functional and/or logical block components or processing steps, which may be performed by any number of hardware, software, and/or firmware components configured to perform the specified functions. For example, certain embodiments employ integrated circuit components, such as memory elements, digital signal processing elements, logic elements, look-up tables, or the like, configured to carry out a variety of functions under the control of one or more processors or other control devices. The connections between functional and logical block components are merely exemplary, which may be direct or indirect, and may follow alternate pathways.

The control system 100 may be a computing system that includes a processing system 110, memory system 120, and input/output (I/O) system 130 for communicating with other devices, such as input devices 99 and output devices 101, either of which may also or alternatively be stored in a cloud 102. The processing system 110 loads and executes an executable program 122 from the memory system 120, accesses data 124 stored within the memory system 120, and directs the system 1 to operate as described in further detail below.

The processing system 110 may be implemented as a single microprocessor or other circuitry, or be distributed across multiple processing devices or sub-systems that cooperate to execute the executable program 122 from the memory system 120. Non-limiting examples of the processing system include general purpose central processing units, application specific processors, and logic devices.

The memory system 120 may comprise any storage media readable by the processing system 110 and capable of storing the executable program 122 and/or data 124. The memory system 120 may be implemented as a single storage device, or be distributed across multiple storage devices or sub-systems that cooperate to store computer readable instructions, data structures, program modules, or other data. The memory system 120 may include volatile and/or non-volatile systems, and may include removable and/or non-removable media implemented in any method or technology for storage of information. The storage media may include non-transitory and/or transitory storage media, including random access memory, read only memory, magnetic discs, optical discs, flash memory, virtual memory, and non-virtual memory, magnetic storage devices, or any other medium which can be used to store information and be accessed by an instruction execution system, for example.

In this manner, the presently disclosed systems and methods provide for a visual cue of the latest investigations that are in progress, but still not updated and available for review by the caregiver. This allows the caregiver to make better clinical decisions with respect to data that is not yet available, but which may be incorporated within the decision making process if results are imminent and can be awaited. While the examples previously described may indicate particular actions by which the caregiver may access the second tier display T2 and third tier display T3 for the visual indicator 30, should be recognized that other mechanisms are also anticipated by the present disclosure. For example, rather than hovering over the icon 31 of the first tier display T1 to access the second tier display T2, a touch screen display device 2 may provide that a first tap on the icon 31 accesses the second tier display T2, whereas two taps brings up the third tier display T3, for example. Shortcuts on the keyboard may also be used to access the various tiers of visual indicators 30, or other mechanisms for cycling between the varying amounts of information relating to the tests that are pending according to the present disclosure.

In addition to providing the caregiver with the essential information described above, the inventor has also recognized that the information provided by the results database 90 may be used for other benefits. For example, the presently disclosed systems and methods provide an indirect way to track lab and diagnosis report turnaround time, based on the estimated completion times 62 discussed above relative to actual completion times. This data may also be used for determining the timeliness of providing care in general.

This written description uses examples to disclose the invention, including the best mode, and also to enable any person skilled in the art to make and use the invention. Certain terms have been used for brevity, clarity, and understanding. No unnecessary limitations are to be inferred therefrom beyond the requirement of the prior art because such terms are used for descriptive purposes only and are intended to be broadly construed. The patentable scope of the invention is defined by the claims and may include other examples that occur to those skilled in the art. Such other examples are intended to be within the scope of the claims if they have features or structural elements that do not differ from the literal language of the claims, or if they include equivalent features or structural elements with insubstantial differences from the literal languages of the claims.

What is claimed is:

1. A method for providing information to a caregiver, the method comprising:
displaying with an interface real-time data for a patient receiving care from the caregiver;
providing access to a results database configured to store results data for the patient;
determining whether a test for the patient is pending, wherein upon completion of the test a test result from the test is stored among the results data in the results database; and
displaying, when the test is determined to be pending, a visual indicator on the interface in addition to the real-time data, wherein the visual indicator provides the information to the caregiver that the test is pending and that the test result corresponding thereto will be accessible via the results database only after the test is completed.

2. The method according to claim 1, wherein pendency of the testing is stored in the results database, and wherein the step of determining whether the testing for the patient is pending includes accessing the results database to identify this pendency of the testing.

3. The method according to claim 1, wherein the test is an unfulfilled test ordered for the patient.

4. The method according to claim 1, wherein the test is a plurality of tests determined to be pending, further comprising determining a quantity of the plurality of tests, wherein the quantity determined is at least part of the visual indicator being displayed.

5. The method according to claim 1, further determining a type of the test determined to be pending, and further comprising displaying the type of the test determined to be pending.

6. The method according to claim 5, wherein the type is one of a plurality of types that include laboratory testing.

7. The method according to claim 5, further comprising displaying the type of the test only when the caregiver at least one of hovers over and clicks on the visual indicator with a selection device.

8. The method according to claim 5, further determining which of a plurality of subtests are included within the test determined to be pending, and further comprising displaying the plurality subtests determined to be pending only when the caregiver clicks on at least one of the visual indicator and the type of the test being displayed with a selection device.

9. The method according to claim 1, further determining a starting time for the test determined to be pending, and further comprising displaying the starting time on the interface.

10. The method according to claim 9, further comprising displaying the starting time only when the caregiver at least one of hovers over and clicks on the visual indicator with a selection device.

11. The method according to claim 1, further determining an expected completion time for the test determined to be pending, wherein the excepted completion time is when the test result is expected to be stored among the results data in the results database, and further comprising displaying the expected completion time on the interface.

12. The method according to claim 11, further determining a type of the test determined to be pending, and further comprising determining the expected completion time based on a standard time for completing the type of test.

13. The method according to claim 11, further comprising displaying the expected completion time only when the caregiver at least one of hovers over and clicks on the visual indicator with a selection device.

14. The method according to claim 11, further comprising comparing the expected completion time to a current time, and further comprising visually modifying the visual indicator based on the comparison of the expected completion time to the current time.

15. The method according to claim 14, wherein the visual indicator includes a colored portion, and wherein the colored portion is modified at least in part depending on the comparison of the expected completion time to the current time.

16. The method according to claim 1, wherein the real-time data includes a heart rate of the patient.

17. The method according to claim 16, wherein the test is x-ray imaging of the patient.

18. The method according to claim 1, wherein the interface concurrently displays additional data relating to other patients.

19. A method for providing information to a caregiver, the method comprising:
displaying with an interface real-time data for a patient receiving care from the caregiver;
providing access to a results database configured to store results data for the patient;
determining a quantity of tests and types of individual tests within the tests pending for the patient, wherein upon completion of each of the individual tests a test result corresponding thereto is stored among the results data in the results database;
displaying, when the quantity of tests pending is determined to be at least one, a visual indicator on the interface in addition to the real-time data, wherein the visual indicator includes the quantity of the tests and types of each of the individual tests therein determined to be pending, wherein the visual indicator provides the information to the caregiver that the test results for each of the individual tests will be accessible via the results database only as each of the individual tests is completed; and
displaying the types of each of the individual tests are displayed only when the caregiver at least one of hovers over and clicks on the visual indicator with a selection device.

20. A method for providing information to a caregiver, the method comprising:
displaying with an interface real-time data for a patient receiving care from the caregiver, wherein the interface is configured to concurrently display additional data corresponding to other patients receiving care from the caregiver;
providing access to a results database configured to store results data for the patient;
determining a quantity of tests and types of individual tests within the tests pending for the patient, wherein upon completion of each of the individual tests a test result corresponding thereto is stored among the results data in the results database;
determining an expected completion time for each of the individual tests determined to be pending, wherein the excepted completion time is when the test result is expected to be stored among the results data in the results database;
displaying, when the quantity of tests pending is determined to be at least one, a visual indicator on the interface in addition to the real-time data, wherein the visual indicator includes the quantity of the tests and types of each of the individual tests therein determined to be pending, wherein the visual indicator provides the information to the caregiver that the test results for each of the individual tests will be accessible via the results database only as each of the individual tests is completed;
displaying the types of each of the individual tests are displayed only when the caregiver at least one of hovers over and clicks on the visual indicator with a selection device; and
displaying the expected completion time for each of the individual tests determined to be pending only when the caregiver with a selection device at least one of: hovers over the visual indicator, clicks on the visual indicator, and clicks on the type corresponding to at least one of the individual tests being displayed on the interface.
